(19) United States
(12) Patent Application Publication
(10) Pub. No.: US 2026/0034341 A1
(43) Pub. Date: Feb. 5, 2026

(54) PLIERS FOR ENGAGING ASEPTIC CONNECTION ASSEMBLY

(71) Applicant: Merck Sharp & Dohme LLC, Rahway, NJ (US)

(72) Inventors: Drew Clifford Porter, Schwenksville, PA (US); Kevin V. Sturtevant, Quakertown, PA (US); John J. Kim, Wilmington, DE (US); Derrick Matthew Smith, Newtown Square, PA (US)

(21) Appl. No.: 18/793,657

(22) Filed: Aug. 2, 2024

(51) Int. Cl. A61M 39/18; B33Y 80/00

(52) U.S. Cl. CPC A61M 39/18; B33Y 80/00; A61M 2209/04

(57) ABSTRACT

Pliers for engaging an aseptic connection assembly is disclosed. The pliers has two arms. Each arm includes a hinge portion, a handle portion, and a pair of phalanges. The hinge portion has a hole for forming a hinge. The handle portion couples to the hinge portion. The pair of phalanges is on a plane perpendicular to the plane of rotation of the two arms and is coupled to the hinge portion. The phalanges couple to sides of outer surfaces of a pair of connector bodies. The hinge is formed by a rod inserted through the holes of the hinge portions of the two arms. An actuation force applied to the handle portions causes the two arms to rotate about the hinge, causing the pair of phalanges to move together to snap together the pair of connector bodies, thereby engaging the aseptic connection assembly.

### BACKGROUND

An aseptic connection assembly is composed of two connector bodies that are snapped together on both sides of the connector bodies. Each connector body includes a hose barb for connecting to tubes and a sealing member that engages with the sealing member of the counterpart connector body. To engage the two connector bodies, a user holds and presses the two connector bodies together. Then the user snaps together the corresponding sides of the connector bodies. Then the user can snap together pull tabs that are attached to protective membranes covering the sealing members. With the snapped together pull tabs, the user can disconnect the pull tabs and protective membranes, thereby unveiling the sealing members to form the aseptic seal.

A tube pinch clamp slides onto a tube through two holes in the pinch clamp. To pinch the tube, a user pushes a first end of the pinch clamp towards a second end that interlocks with the first end. As the two ends are interlocked, internal protrusions positioned on the pinch clamp on either side of the engaged tube pinch the tube closed.

### SUMMARY

In one or more embodiments, pliers aid a user in engaging an aseptic connection assembly. The pliers include two arms, with each arm including a hinge portion, a handle portion, and a pair of phalanges. The hinge portion has a first hole for forming a hinge. The hinge is formed by a rod inserted through the holes of the hinge portions of the two arms. The handle portion couples to and extends radially from the hinge portion. The pair of phalanges is disposed on a plane perpendicular to the plane of rotation of the two arms and is coupled to the second end of the hinge portion. The phalanges couple to sides of outer surfaces of a pair of connector bodies. An actuation force applied to the handle portions causes the two arms to rotate about the hinge and the pair of phalanges to move towards one another to snap together the pair of connector bodies, thereby forming the aseptic connection assembly.

In one or more embodiments, pliers aid a user in engaging a tubing pinch clamp. The pliers include two arms, with each arm including a hinge portion, a handle portion, and a gripping portion. The hinge portion has a first hole for forming a hinge. The hinge is formed by a rod inserted through the holes of the hinge portions of the two arms. The handle portion couples to and extends radially from the hinge portion. The gripping portion is coupled to the second end of the hinge portion. The gripping portions couple to outer surfaces of a hook portion and a barb portion of the tubing pinch clamp. An actuation force applied to the handle portions causes the two arms to rotate about the hinge and the gripping portions to move towards one another to lock the barb portion to the hook portion, thereby pinching the tubing.

### BRIEF DESCRIPTION OF THE DRAWINGS

FIG. 1 illustrates a perspective view of an aseptic connection assembly with connector bodies decoupled, in accordance with one or more embodiments.

FIG. 2 illustrates a perspective view of pliers for engaging an aseptic connection assembly, according to one or more embodiments.

FIG. 3 illustrates a perspective view of the pliers of FIG. 2 engaging connector bodies of the aseptic connection assembly of FIG. 1, according to one or more embodiments.

FIG. 4 illustrates a perspective view of a tubing pinch clamp, according to one or more embodiments.

FIG. 5 illustrates a perspective view of pliers for engaging the tubing pinch clamp, according to one or more embodiments.

FIG. 6 illustrates a method flowchart for a process of manufacturing pliers, according to one or more embodiments.

The figures depict various embodiments of the present invention for purposes of illustration only. One skilled in the art will readily recognize from the following discussion that alternative embodiments of the structures and methods illustrated herein may be employed without departing from the principles of the invention described herein.

### DETAILED DESCRIPTION

### Aseptic Connection Assembly

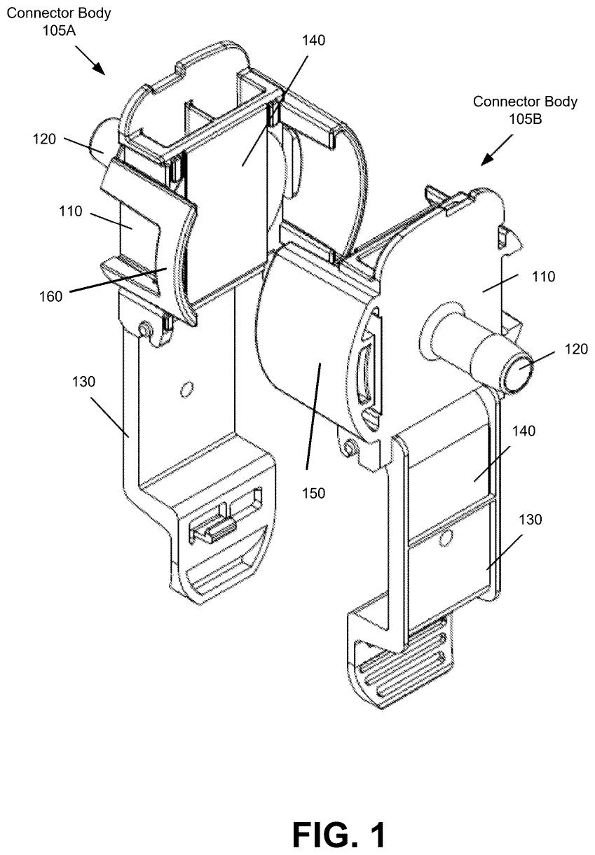

FIG. 1 illustrates a perspective view of an aseptic connection assembly 100 with connector bodies 105 decoupled, in accordance with one or more embodiments. The aseptic connection assembly 100 provides an aseptic connection to two tubes (also referred to as hoses), ensuring sterile transfer of fluid.

For example, the aseptic connection assembly 100 can be used in the pharmaceutical, biotech, and food industries to transfer sterile fluids without the risk of contamination. This is achieved by securely joining two separate sterile systems in a manner that does not allow microorganisms, particles, or impurities from the environment to enter. Such connections maintain the sterility of products, equipment, or processes. This is particularly important in the production of injectable drugs, vaccines, or sterile food products where contamination can lead to severe health hazards.

In one or more embodiments, the aseptic connection assembly 100 includes two connector bodies 105, connector body 105A and connector body 105B. The two connector bodies 105 are coupled together forming a sealed channel for transferring sterile fluid through the aseptic connection assembly 100. FIG. 1 shows the two connector bodies 105 in an uncoupled state.

Each connector body 105 includes a main body 110 with a conduit connection 120 extending from the main body 110. The main body 110 may form a planar outer surface opposite to an inner surface with a central indent. The inner surfaces of the two connector bodies 105 couple together when the two connector bodies 105 are in the coupled state, forming a fully enclosed cavity. The indentation on the inner surface of a connector body may be formed by a circular escarpment. The conduit connection 120 extends perpendicularly through the main body, i.e., extends perpendicularly away from the outer surface and extends perpendicularly away from the inner surface. The conduit connection 120 may have a hollow cylindrical shape and may be sized to couple to a hose or tube. The conduit connection 120 extends from the indentation of the inner surface to the coupling plane of the two inner surfaces of the connector bodies 105.

Each connector body 105 may further include a protective cover 130 removably attached to the main body 110. As shown in FIG. 1, the protective cover 130 is hinged to the main body. In a packaged state (e.g., as prepared by a manufacturer in a sterile environment), the protective cover 130 is rotated upward (i.e., 180 degrees) about the hinge to cover the inner surface of the main body 110. This provides a layer of protection from contaminants reaching the conduit connection 120 extending from the inner surface of the main body 110. The protective cover 130 may be further attached to a protective membrane 140 that also covers the inner surface of the connector body 105. The protective membrane 140 may be attached at one or more positions around the inner surface of the main body 110. The protective membrane 140 provides an added layer of protection from contaminants reaching the portion of the conduit connection 120 extending from the inner surface of the main body 110. The protective cover 130 of each connector body 105 may include a clip that interlocks with a counterpart structure (e.g., a hole) on another connector body 105.

The connector body 105 may further include one or more connectors for locking the two connector bodies 105 together when in the coupled state. In one or more embodiments, each connector body 105 includes a first connector 150 and a second connector 160. In the embodiment shown, the first connector 150 and the second connector 160 are located on opposite sides of the main body 110. The first connector 150 is designed to be engaged with the second connector 160, ensuring proper alignment of the inner surfaces of the connector bodies 105. Accordingly, when the first connector body 105A is oriented with its inner face facing the inner face of the second connector body 105B, the second connector 160 of the first connector body 105A will engage with the first connector 150 of the second connector body 105B. Likewise, the second connector 160 of the second connector body 105B will engage with the first connector 150 of the first connector body 105A. Many types of connectors that perform the functions of engagement and alignment may be utilized without departing from the concepts presented herein. To engage the corresponding connectors, a user presses firmly against the outer surfaces of the main bodies of the two connector bodies 105. In some embodiments, the connectors may be structured to provide a clicking sound when the two connectors are fully engaged and locked. When a human operator is manually engaging the connectors (e.g., without the aid of any tool), the human operator would engage connectors on one side of the connector bodies 105, with a first audible click, then engage connectors on the other side of the connector bodies 105, with a second audible click.

In the embodiment shown, the first connector 150 includes an extension with a curved main portion (e.g., a quarter circle arc) and side portions (e.g., pointing inwards along a chord of the circle formed by the quarter circle arc) that together form an open channel. The channel defines an interior volume within which a latch member is positioned. The second connector 160 includes an extension with a curved main portion and side edges connecting the curved main portion to the main body. The curved main portion of the second connector 160 is configured to be key into the interior volume of the channel formed by the first connector 150. Proper alignment between two connector bodies 105 is ensured by the side portions of the first connector 150 which surround and guide the side edges of the second connector 160. In the embodiment shown, on an outer surface of the main body, connector 150 includes one or more holes on the outer surface, whereas connector 160 includes an indent between the two side edges. In one or more embodiments, the first connectors 150 of the connector bodies 105 are identical and/or the second connectors 160 of the connector bodies 105 are identical. In one or more embodiments, the two connector bodies are identical.

In one or more embodiments, to engage the two connector bodies, a user unpackages both connector bodies 105. The user connects each connector body 105's conduit connection 120 to a hose or tube. The user rotates open the protective cover 130 revealing the protective membrane 140 on each connector body. The user aligns the two connector bodies 105, such that the first connector 150 of each connector body 105 is aligned with the second connector 160 of the other connector body 105. The user presses the two connector bodies 105 together. The user presses on the outer surfaces of the two connector bodies 105 on one side of the main body 110, to lock the first connector and the second connector together. The user presses on the outer surfaces of the two connector bodies 105 on the opposite side of the main body 110 to lock the other first connector and the other second together. The user presses the two protective covers 130 together to lock them together. The user pulls the protective covers 130 away from main bodies 110, thereby removing the protective membranes 140 and forming the aseptic connection between the conduit connections 120 disposed within the inner cavity formed by the joined inner surfaces.

### Pliers for Engaging Aseptic Connection Assembly

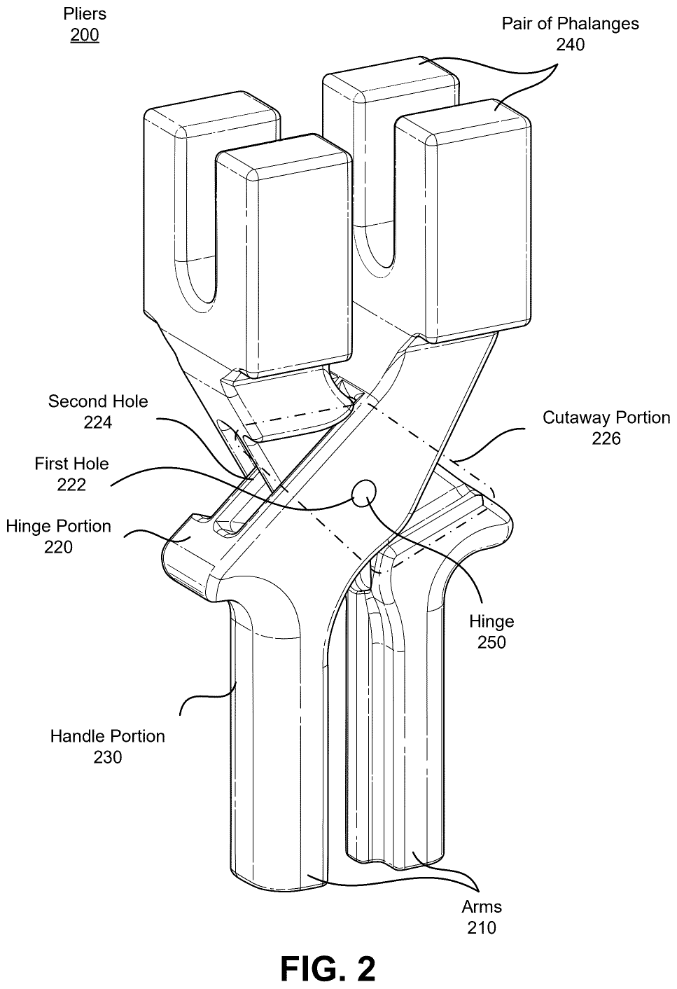

FIG. 2 illustrates a perspective view of the pliers 200 for engaging connector bodies of the aseptic connection assembly of FIG. 1, according to one or more embodiments.

The pliers 200 include two arms 210 coupled at a hinge 250. The two arms rotate about one another at the hinge 250. Each arm 210 includes a hinge portion 220, a handle portion 230, and a pair of phalanges 240. A user holds the pliers 200 at the handle portions 230 and engages the pairs of phalanges 240 to the connector bodies of the aseptic connection assembly. As the user actuates the pliers 200, i.e., applying a force to bring the handle portions closer together, the pairs of phalanges 240 are likewise brought closer together to couple the two connector bodies of the aseptic assembly together.

Each hinge portion 220 includes a first hole 222 disposed in an intermediate point of the hinge portion. The hinge portion 220 includes two ends, a first end and a second end. The first hole 222 is disposed in between the first end and the second end. The first end of the hinge portion 220 couples to the handle portion 230, whereas the second end of the hinge portion 220 couples to the pair of phalanges 240. In one or more embodiments, the pair of phalanges 240 and the handle portion 230 are substantially parallel, and the hinge portion 220 is coupled to the handle portion 230 and the pair of phalanges 240 at obtuse angles. The hinge 250 is formed with a rod (linear) inserted through the aligned holes 222 of the hinge portions 220. The first holes 222 are perpendicular to the plane formed by the parallel handle portion 230 and the pair of phalanges 240.

In one or more embodiments, the hinge portions 230 of the two arms 210 include second holes 224 that are perpendicular to the first holes 222. The second holes 224 are larger than the first holes 222 and may be of a trapezoidal prism shape with small depth. The hinge portions 220 can interlink with one another via the second holes 224. In such embodiments, the two arms 210 of the pliers 200 may be three-dimensional (3D) printed concurrently to interlink the hinge portions 220.

Each hinge portion 220 may also include a cutaway portion 226 with two escarpments to limit the range of rotation between the two arms 210. The cutaway portion 226 runs parallel to the second 224, having a similar hollow volume to the second hole 224. In one or more embodiments, the hinge 250 is a spring-loaded hinge to bias the two arms 210 away from one another, increasing the angle between the handle portions 230 and the pairs of phalanges 240, respectively. The escarpments of the cutaway portions 226 provide a stopping point from rotation of the two arms 210, i.e., a maximum angle in the range of rotation of the two arms 210 about the hinge 250, whereas a minimum angle is determined by contact of the handle portions 230 and/or contact of the pairs of phalanges 240.

The pair of phalanges 240 on one arm 210 engage to the connector bodies to couple the connector bodies of the aseptic connection assembly together. Each pair of phalanges 240 may be disposed on a plane perpendicular to the plane of rotation of the two arms. The inner surfaces of the pair of phalanges 240 on one arm face the inner surfaces of the pair of phalanges 240 on the other arm. The inner surfaces are configured to couple to the outer surfaces of the connector bodies. Accordingly, the inner surfaces of the pair of phalanges 240 is planar. In some embodiments, one or both phalanges 240 includes a protrusion that extends perpendicularly from the plane formed by the inner surface of the phalanges 240. The protrusion may be sized to key into an indent on the outer surface of the connector bodies. The indent aids in aligning the position of the connector body relative to the pair of phalanges 240. In some embodiments, each pair of phalanges 240 includes the protrusion to key into the indent of their respective connector body. The phalanges 240 on one arm 210 are distanced to fit the conduit connection of the connector body.

The hinge 250 aids in coupling the two arms 210 of the pliers 200. The hinge is formed by a rod inserted through the first holes 222 of the hinge portions 220 of the arms 210. The hinge 250 may further be spring-loaded, to bias the arms 210 thereby increasing their angular distance about the hinge 250. In one or more embodiments, the spring-loaded hinge 250 may be formed from a compression spring having ends coupled to inner surfaces of the handle portions 230 of the two arms 210 facing one another. In other embodiments, the spring-loaded hinge 250 may be formed from a torsion spring having a coil disposed around the rod of the hinge and legs extending from the coil coupled to inner surfaces of the handle portions of the two arms facing one another.

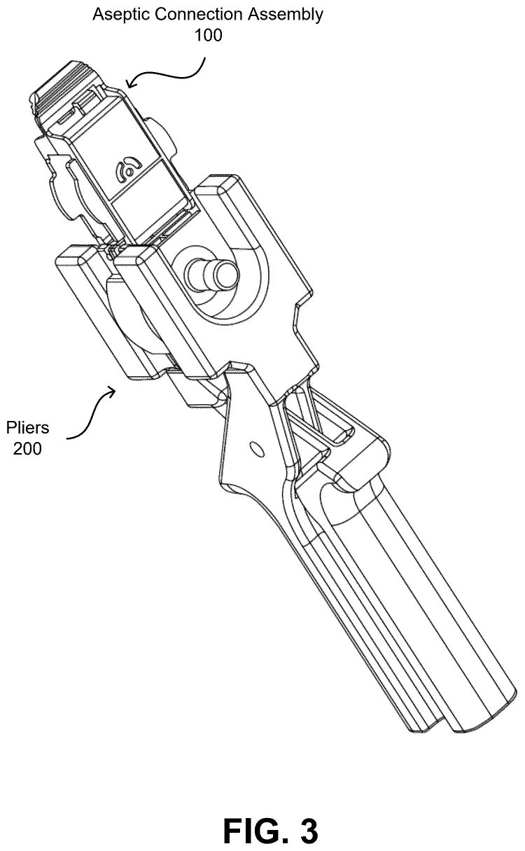

FIG. 3 illustrates a perspective view of the pliers 200 of FIG. 2 engaging the aseptic connection assembly of FIG. 1, according to one or more embodiments. The connector bodies of the aseptic connection assembly 100 are aligned together, e.g., by one hand of a user. The pliers 200 are engaged to the connector bodies of the aseptic connection assembly 100. In particular, the user may grip the handle portions of the pliers 200 and position the phalanges such that the inner surfaces of the phalanges contact the outer surfaces of the connector bodies. In positioning the pliers 200, the user may also aim to align the conduit connection of each connector body in between a respective pair of phalanges. The user actuates the handle portions, e.g., squeezing the handle portions together with the gripping hand. As the handle portions are brought close together, i.e., decreasing an angular distance, the pairs of phalanges on the two arms likewise are brought together, i.e., decreasing their angular distance. The actuation force translates into force pressing on the outer surfaces of the connector bodies to couple the connector bodies together. In some embodiments, the connector bodies provide the audio feedback when securely coupled together. With the aid of the pliers 200 of FIG. 2, in embodiments with each connector body including two connectors, the actuation of the pliers 200 to engage the connector bodies would yield a single audible click as the two sets of connectors are engaged and locked contemporaneously. In some embodiments with the protective membrane, the user may finish installation of the aseptic connection by removal of the pull tabs connected to the protective membrane. The result is an installed aseptic connection assembly 100 with an aseptic connection between two aseptic conduits.

### Tube Pinch Clamp

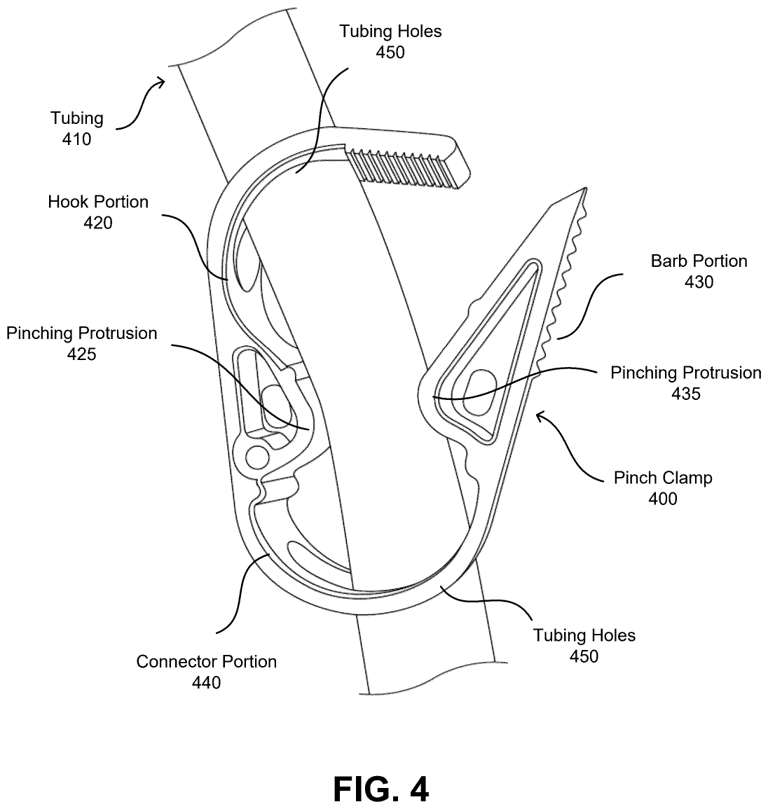

FIG. 4 illustrates a perspective view of a tube pinch clamp 400 on tubing 410, according to one or more embodiments. The pinch clamp 400 is monolithic, formed of a rigid material with some compliance. In some embodiments, the pinch clamp is formed from a thermoplastic, metal, metal alloys, etc.

The pinch clamp 400 includes a hook portion 420 and a barb portion 430 coupled at a connector portion 440. In particular, the hook portion 420 is coupled to a first end of the connector portion 440, having a semi-circular shape, and the barb portion 430 is coupled to a second end of the connector portion 440, opposite the first end. In a disengaged position (equilibrium state), a free end of the hook portion 420 and a free end of the barb portion 430 are distant and not in contact. An inner surface of the free end of the hook portion 420, i.e., along the curved formed by the smaller radius of the hook, may include a grated surface. To engage the pinch clamp 400, a user squeezes the hook portion 420 and the barb portion 430 together, such that the free end of the barb portion 430 engages with the grated inner surface of the free end of the hook portion 420. The grated inner surface locks the barb portion 430 in place, forming a t-shape between the hook portion 420 and the barb portion 430. The connector portion 440 is under tension and/or compression in the engaged state, i.e., an outer surface is under tension, while an inner surface is under compression. To disengage the pinch clamp 400, the user lifts the hook portion 420 away from the barb portion 430, i.e., releasing the grated inner surface of the hook portion 420 from the barb portion 430. The connector portion 440's tension and/or compression causes the barb portion 430 to return to the disengaged position (i.e., equilibrium state as shown in FIG. 4).

The pinch clamp 400 further includes two tubing holes 450 for sliding the pinch clamp 400 onto the tubing 410. The first hole is positioned in the hook portion 420, perpendicular to the curve. The second hole is positioned in the connector portion 440. The two holes may be sized to appropriately fit the tubing 410.

To pinch the tubing 410, the hook portion 420 includes a pinching protrusion 425 and the barb portion includes a pinching protrusion 435. In the engaged state, the two pinching protrusions 425 and 435 are brought close together (i.e., compared to the disengaged state). The proximity of the two pinching protrusions 425 and 435 squeeze the tubing 410 from opposite sides, thereby impeding fluid flow through the tubing 410.

### Pliers for Engaging Tube Pinch Clamp

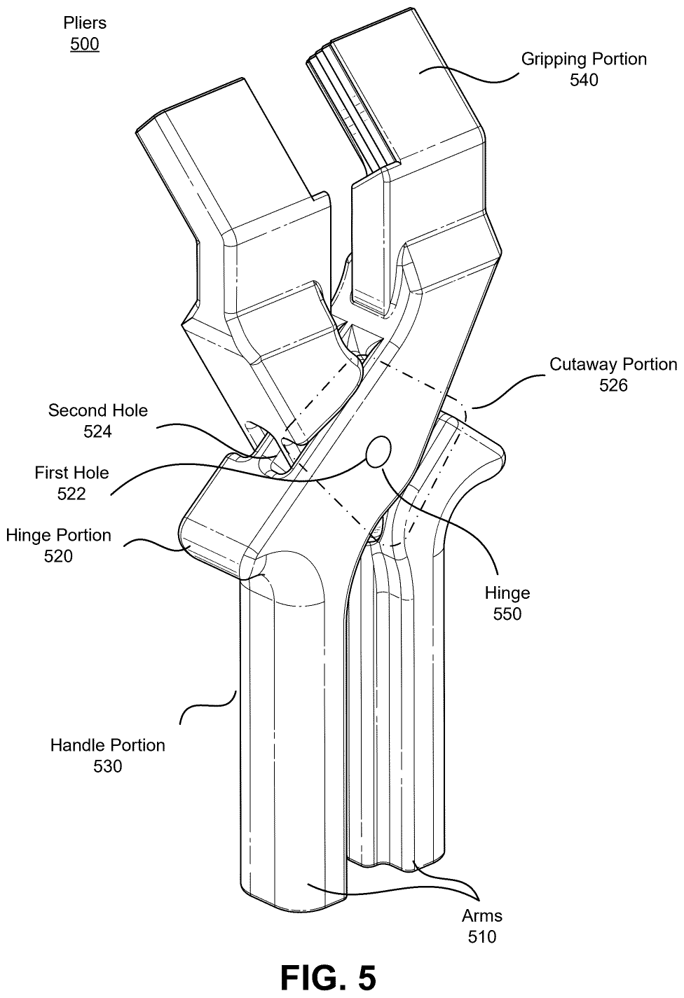

FIG. 5 illustrates a perspective view of pliers 500 for engaging the pinch clamp of FIG. 4, according to one or more embodiments.

The pliers 500 include two arms 510 coupled at a hinge 550. The two arms rotate about one another at the hinge 550. Each arm 510 includes a hinge portion 520, a handle portion 530, and a gripping portion 540. A user holds the pliers 500 at the handle portions 530 and engages the gripping portions 540 to the outer surfaces of the hook portion and the barb portion of a pinch clamp. As the user actuates the pliers 500, i.e., applying a force to bring the handle portions closer together, the gripping portions 540 are likewise brought closer together to lock the barb portion to the hook portion.

Each hinge portion 520 includes a first hole 522 disposed in an intermediate point of the hinge portion. The hinge portion 520 includes two ends, a first end and a second end. The first hole 522 is disposed in between the first end and the second end. The first end of the hinge portion 520 couples to the handle portion 530, whereas the second end of the hinge portion 520 couples to the gripping portion 540. In one or more embodiments, the gripping portion 540 and the handle portion 530 are substantially parallel, and the hinge portion 520 is coupled to the handle portion 530 and the gripping portion 540 at obtuse angles. The hinge 550 is formed with a rod (linear) inserted through the aligned holes 522 of the hinge portions 520. The first holes 522 are perpendicular to the plane formed by the parallel handle portion 530 and the gripping portion 540.

In one or more embodiments, the hinge portions 530 of the two arms 510 include second holes 524 that are perpendicular to the first holes 522. The second holes 524 are larger than the first holes 522 and may be of a trapezoidal prism shape with small depth. The hinge portions 520 can interlink with one another via the second holes 524. In such embodiments, the two arms 510 of the pliers 500 may be three-dimensional (3D) printed concurrently to interlink the hinge portions 520.

Each hinge portion 520 may also include a cutaway portion 526 with two escarpments to limit the range of rotation between the two arms 510. The cutaway portion 526 runs parallel to the second 524, having a similar hollow volume to the second hole 524. In one or more embodiments, the hinge 550 is a spring-loaded hinge to bias the two arms 510 away from one another, increasing the angle between the handle portions 530 and the pairs of phalanges 540, respectively. The escarpments of the cutaway portions 526 provide a stopping point from rotation of the two arms 510, i.e., a maximum angle in the range of rotation of the two arms 510 about the hinge 550, whereas a minimum angle is determined by contact of the handle portions 530 and/or contact of the pairs of phalanges 540.

The gripping portions 540 on the arms 510 engage the outer surfaces of the hook portion and the barb portion of the pinch clamp to engage the pinch clamp. Each gripping portion 540 may be disposed on a plane perpendicular to the plane of rotation of the two arms. The inner surface of the gripping portion 540 on one arm faces the inner surface of the gripping portion 540 on the other arm. The inner surfaces are configured to couple to the outer surfaces of the pinch clamp, i.e., on either side of the tubing. Accordingly, the inner surfaces of the gripping portion 540 is planar. In some embodiments, the inner surfaces of the gripping portions 540 are grated to better grip to the outer surfaces of the pinch clamp. In such embodiments, the gripping portions 540 include linear grooves aligned radially from the hole of the hinge 550. In other embodiments, the inner surfaces of the gripping portions 540 may be coated with a non-slip material, e.g., rubber, grip tape, etc. In some embodiments, the gripping portion 540 of each arm 510 includes an escarpment abutting the linear grooves and positioned at an intermediate point of the gripping portion. The escarpment aids in preventing slippage along the direction of the linear grooves. In other embodiments, the gripping portion 540 of each arm 510 comprises a first portion and a second portion. The first portion couples to the hinge portion 520, wherein the second portion couples to the first portion at an obtuse angle in a plan perpendicular to the plane of rotation of the two arms 510 about the hinge 550. The second portions of the two gripping portions 540 are parallel. The angled gripping portions 540 provide ease of coupling to the pinch clamp, e.g., for improved ergonomics and/or reducing slippage between the pliers 500 and the pinch clamp.

The hinge 550 aids in coupling the two arms 510 of the pliers 500. The hinge is formed by a rod inserted through the first holes 522 of the hinge portions 520 of the arms 510. The hinge 550 may further be spring-loaded, to bias the arms 510 thereby increasing their angular distance about the hinge 550. In one or more embodiments, the spring-loaded hinge 550 may be formed from a compression spring having ends coupled to inner surfaces of the handle portions 530 of the two arms 510 facing one another. In other embodiments, the spring-loaded hinge 550 may be formed from a torsion spring having a coil disposed around the rod of the hinge and legs extending from the coil coupled to inner surfaces of the handle portions of the two arms facing one another.

### Example Construction

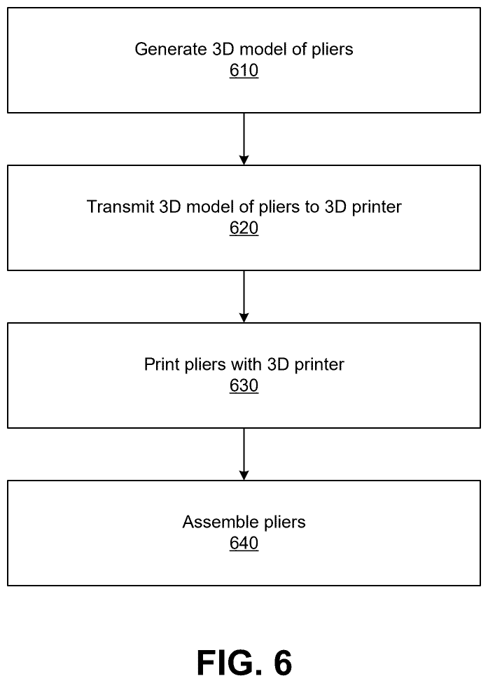

FIG. 6 illustrates a method flowchart for a process of manufacturing pliers (e.g., the pliers 200 and/or the pliers 500), according to one or more embodiments. Some or all of the steps may be automated by one or more machines.

The process includes generating 610 a 3D model of the pliers. The 3D model may include spatial dimensions of the pliers. The 3D model may further include printing support structures that aid in the 3D printing process. Such support structures may be removed post-3D-printing.

The process includes transmitting 620 the 3D model of the pliers to a 3D printer. The 3D printer can determine the print instructions based on the 3D model. For example, the print instructions may include an orientation to print the pliers, speed of the printing, movement of the print head, etc. Different 3D printing approaches may be implemented, including: stereolithography 3D printing, selective laser sintering 3D printing, multi jet fusion 3D printing, fused deposition modeling 3D printing, etc.

The process includes printing 630 the pliers with the 3D printer. Based on the print instruction and/or the 3D model, the 3D printer crafts the pliers from unformed material (e.g., either in liquid, powder, or solid form) in a layer-by-layer printing process. Once printed, the pliers may be cured, coated, sanded, or any other post-printing finishing process.

The process includes assembling 640 the pliers. In some embodiments, the two arms of the pliers may be 3D-printed separately. In other embodiments, the two arms of the pliers may be 3D-printed concurrently, e.g., to be interlocked at the hinge portions. To join the two arms, a rod and/or spring is used to join the two pliers. For example, the rod may be inserted into holes of the arms to join the two at the hinge. In other examples, a torsion spring is inserted into the holes of the arms with hinges of the torsion spring coupled lengthwise to the arms. In some embodiments, the pliers may be further subdivided into constituent parts. In such embodiments, each constituent part may be separately printed. Assembly may include adhering the parts together, e.g., with welding, glue, etc.

### ADDITIONAL CONSIDERATIONS

The foregoing description of the embodiments has been presented for the purpose of illustration; many modifications and variations are possible while remaining within the principles and teachings of the above description.

Any of the steps, operations, or processes described herein may be performed or implemented with one or more hardware or software modules, alone or in combination with other devices. In some embodiments, a software module is implemented with a computer program product comprising one or more computer-readable media storing computer program code or instructions, which can be executed by a computer processor for performing any or all of the steps, operations, or processes described. In some embodiments, a computer-readable medium comprises one or more computer-readable media that, individually or together, comprise instructions that, when executed by one or more processors, cause the one or more processors to perform, individually or together, the steps of the instructions stored on the one or more computer-readable media. Similarly, a processor may comprise one or more subprocessing units that, individually or together, perform the steps of instructions stored on a computer-readable medium.

Embodiments may also relate to a product that is produced by a computing process described herein. Such a product may store information resulting from a computing process, where the information is stored on a non-transitory, tangible computer-readable medium and may include any embodiment of a computer program product or other data combination described herein.

Where values are described as “approximate” or “substantially” (or their derivatives), such values should be construed as accurate+/−10% unless another meaning is apparent from the context. From example, “approximately ten” should be understood to mean “in a range from nine to eleven.”

As used herein, the terms “comprises,” “comprising,” “includes,” “including,” “has,” “having,” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive “or” and not to an exclusive “or”. For example, a condition “A or B” is satisfied by any one of the following: A is true (or present) and B is false (or not present); A is false (or not present) and B is true (or present); and both A and B are true (or present). Similarly, a condition “A, B, or C” is satisfied by any combination of A, B, and C being true (or present). As a not-limiting example, the condition “A, B, or C” is satisfied when A and B are true (or present) and C is false (or not present). Similarly, as another not-limiting example, the condition “A, B, or C” is satisfied when A is true (or present) and B and C are false (or not present).

The language used in the specification has been principally selected for readability and instructional purposes, and it may not have been selected to narrow the inventive subject matter. It is therefore intended that the scope of the patent rights be limited not by this detailed description, but rather by any claims that issue on an application based hereon.

What is claimed is:

1. Pliers for engaging an aseptic connection assembly, the pliers comprising:
first and second arms, each arm comprising:
a hinge portion comprising a first end, a second end opposite the first end, and a first hole disposed in an intermediate point of the hinge portion between the first end and the second end;
a handle portion coupled to the first end of the hinge portion extending radially from the hole of the hinge portion in a plane of rotation of the two arms about the holes; and
a pair of phalanges disposed on a plane perpendicular to the plane of rotation of the two arms about the holes and coupled to the second end of the hinge portion, each phalange extending radially from the hole of the hinge portion in the plane of rotation of the two arms about the holes, wherein an inner surface of the pair of phalanges is configured to couple to both sides of an outer surface of one connector body of a pair of connector bodies forming the aseptic connection assembly; and
a hinge formed by a rod inserted through the holes of the hinge portions of the two arms, wherein an actuation force applied to the handle portions of the two arms causes the two arms to rotate about the hinge and the pair of phalanges to move towards one another to snap together the pair of connector bodies forming the aseptic connection assembly.

2. The pliers of claim 1, wherein the handle portion and the hinge portion form an obtuse angle, wherein the pair of phalanges and the hinge portion from an obtuse angle, and wherein the handle portion and the pair of phalanges are parallel.

3. The pliers of claim 1, wherein the hinge portion of each arm includes a second hole perpendicular to the first hole, and wherein the hinge portions of the arms are interlinked through the second holes.

4. The pliers of claim 3, wherein the two arms are three-dimensional (3D) printed concurrently to interlink the second holes of the hinge portions.

5. The pliers of claim 1, wherein each hinge portion includes a cutaway portion with two escarpments that limit a range of rotation of the two arms about the hinge.

6. The pliers of claim 1, wherein the phalanges of each arm are parallel to one another.

7. The pliers of claim 1, wherein the phalanges of each arm are positioned equidistant from a bisection plane lengthwise bisecting the rod of the hinge.

8. The pliers of claim 1, wherein the inner surface of each pair of phalanges is planar.

9. The pliers of claim 1, wherein a first phalange in each pair of phalanges comprises a protrusion coupled to the inner surface to key into an indent on the outer surface of the corresponding connector body.

10. The pliers of claim 1, wherein the hinge is a spring-loaded hinge.

11. The pliers of claim 10, wherein the spring-loaded hinge is a compression spring having ends coupled to inner surfaces of the handle portions of the two arms facing one another.

12. The pliers of claim 10, wherein the spring-loaded hinge is a torsion spring having a coil disposed around the rod of the hinge and legs extending from the coil coupled to inner surfaces of the handle portions of the two arms facing one another.

13. A method for manufacturing pliers for engaging an aseptic connection assembly, the method comprising:
transmitting a three-dimensional (3D) model for the pliers to be printed on a 3D printer, the 3D model for the pliers comprising:
first and second arms, each arm comprising:
a hinge portion comprising a first end, a second end opposite the first end, and a first hole disposed in an intermediate point of the hinge portion between the first end and the second end,
a handle portion coupled to the first end of the hinge portion extending radially from the hole of the hinge portion in a plane of rotation of the two arms about the holes, and
a pair of phalanges disposed on a plane perpendicular to the plane of rotation of the two arms about the holes and coupled to the second end of the hinge portion, each phalange extending radially from the hole of the hinge portion in the plane of rotation of the two arms about the holes, wherein an inner surface of the pair of phalanges is configured to couple to both sides of an outer surface of one connector body of a pair of connector bodies forming the aseptic connection assembly
fabricating the two arms using the 3D printer; and
joining the two arms with a hinge by inserting a rod through the holes of the hinge portions of the two arms, wherein an actuation force applied to the handle portions of the two arms causes the two arms to rotate about the hinge and the pair of phalanges to move towards one another to snap together the pair of connector bodies forming the aseptic connection assembly.

14. The method of claim 13, wherein fabricating the two arms using the 3D printer comprises concurrently fabricating the two arms to interlink the second holes of the hinge portions.

15. Pliers comprising:
first and second arms, each arm comprising:
a hinge portion comprising a first end, a second end opposite the first end, and a first hole disposed in an intermediate point of the hinge portion between the first end and the second and a second hole perpendicular to the first hole;
a handle portion coupled to the first end of the hinge portion extending radially from the hole of the hinge portion in a plane of rotation of the two arms about the holes; and
a gripping portion coupled to the second end of the hinge portion extending radially from the hole of the hinge portion; and
a hinge formed by a rod inserted through the holes of the hinge portions of the two arms, wherein an actuation force applied to the handle portions of the two arms causes the two arms to rotate about the hinge and the gripping portions to move towards one another,
wherein the hinge portions of the arms are interlinked through the second holes, and wherein the two arms are three-dimensional (3D) printed concurrently to interlink the second holes of the hinge portions.

16. The pliers of claim 15, wherein an inner surface of the gripping portion of each arm includes linear grooves aligned radially from the hole of the hinge portion, wherein the inner surfaces of the gripping portions of the two arms face one another.

17. The pliers of claim 16, wherein the gripping portion of each arm includes an escarpment abutting the linear grooves and positioned at an intermediate point of the gripping portion.

18. The pliers of claim 15, wherein the gripping portion of each arm comprises a first portion and a second portion, wherein the first portion couples to the hinge portion, wherein the second portion couples to the first portion at an obtuse angle in a plan perpendicular to the plane of rotation of the two arms about the holes, and wherein the second portions of the two arms are parallel.

19. The pliers of claim 15, wherein each hinge portion includes a cutaway portion with two escarpments that limit a range of rotation of the two arms about the hinge.

20. The pliers of claim 15, wherein the hinge is a spring-loaded hinge comprising a compression spring having ends coupled to inner surfaces of the handle portions of the two arms facing one another.
